(19) United States
(12) Patent Application Publication
(10) Pub. No.: US 2026/0188488 A1
(43) Pub. Date: Jul. 2, 2026

(54) METHOD FOR MEASURING DNA METHYLATION LEVEL, DISEASE PREDICTION SYSTEM, AND TEST SYSTEM

(71) Applicant: Hitachi, Ltd., Chiyoda-ku, Tokyo (JP)

(72) Inventor: Tomoakira KAWAI, Tokyo (JP)

(21) Appl. No.: 18/858,233

(22) PCT Filed: Jan. 30, 2023
(86) PCT No.: PCT/JP2023/002946

§ 371 Date: Oct. 18, 2024

(30) Foreign Application Priority Data
May 27, 2022 (JP) 2022-086734

(51) Int. Cl. G16H 50/20; G16B 20/20; G16B 40/20; G16H 15/00

(52) U.S. Cl. CPC G16H 50/20; G16B 20/20; G16B 40/20; G16H 15/00

(57) ABSTRACT

The lengthening of measurement and the increase in measurement cost can be prevented by narrowing down the locations (coordinates) where the DNA methylation level is to be measured. A DNA methylation level measurement method includes: performing machine learning using a training data set including a measurement result of a DNA methylation level at a plurality of coordinates and information on a disease corresponding to the measurement result to generate a learning model configured to predict a disease; selecting one or a plurality of principal components of the measurement result (S903); calculating a factor loading indicating a correlation between the selected one or plurality of principal components and the plurality of coordinates (S904); and extracting, based on the calculated factor loading, a coordinate (item) to be measured in a test for measuring the DNA methylation level, from among the plurality of coordinates (S905).

### TECHNICAL FIELD

The present disclosure relates to a DNA methylation level measurement method, a disease prediction system, and a test system.

### BACKGROUND ART

The DNA methylation is a reaction of adding a methyl group to one of bases of a DNA strand, and the DNA methylation is known as a mechanism of inactivation of gene expression. The DNA methylation is suggested to be deeply involved not only in cancerization but also in various diseases such as lifestyle diseases. Accordingly, various techniques for predicting a disease based on the degree of DNA methylation have been proposed (see, for example, PTLs 1 to 3).

### CITATION LIST

### Patent Literature

PTL 1: US2022/0002808
PTL 2: WO2021/202351
PTL 3: JP2019-193578A

### SUMMARY OF INVENTION

### Technical Problem

In PTL 1, Alzheimer's disease is diagnosed by machine learning based on the degree of methylation in one or a plurality of Alzheimer's indicator genes in a blood sample. In PTL 2, colorectal cancer is identified based on the degree of methylation of a cell-free DNA using machine learning. In addition, in PTL 3, identification using an identifier of machine learning, which is characterized by the degree of methylation of two cytosines in a genomic DNA, is performed, and the presence or absence of depression occurring before the age of 50 is predicted.

However, a relationship between a disease and the DNA methylation has not been clarified, and thus methylation measurement in a wide range (for example, about 30,000 genes) is required, and a large amount of cost caused by sequence processing is generated.

Accordingly, the disclosure provides a method for measuring a DNA methylation level, a disease prediction system, and a test system, which prevents an increase in measurement cost and lengthening of measurement in a test for measuring a DNA methylation level by narrowing down locations (coordinates) where a DNA methylation level is to be measured.

### Solution to Problem

A DNA methylation level measurement method according to the disclosure includes: performing machine learning using a training data set including a measurement result of a DNA methylation level at a plurality of coordinates and information on a disease corresponding to the measurement result to generate a learning model configured to predict a disease; selecting one or a plurality of principal components of the measurement result; calculating a degree of correlation indicating a correlation between the selected one or plurality of principal components and the plurality of coordinates; and determining, based on the calculated degree of correlation, a coordinate to be measured in a test for measuring the DNA methylation level, from among the plurality of coordinates.

### Advantageous Effects of Invention

According to the disclosure, by narrowing down the locations (coordinates) where the DNA methylation level is to be measured, the lengthening of the measurement and the increase in the measurement cost can be reduced in the test for measuring the DNA methylation level.

### BRIEF DESCRIPTION OF DRAWINGS

FIG. 1 is a diagram showing an overall configuration of a test system.

FIG. 2 is a diagram showing a graphical user interface (GUI) of the test system.

FIG. 3 is a software block diagram of a disease prediction system.

FIG. 4A is a diagram showing a hardware structure of the test system.

FIG. 4B is a hardware block diagram of a computer system of the disease prediction system.

FIG. 5 is a flowchart of disease prediction by the disease prediction system.

FIG. 6 is a flowchart of disease diagnosis by an HIS.

FIG. 7 is a flowchart of dimensionality compression by the disease prediction system.

FIG. 8 is a flowchart showing an example of learning by the disease prediction system.

FIG. 9 is a flowchart showing an example of dimensionality compression by the disease prediction system.

FIG. 10 is a diagram showing an example of each data.

FIG. 11 is a graph showing a cumulative contribution ratio.

FIG. 12 is a graph showing a factor loading.

### DESCRIPTION OF EMBODIMENTS

Embodiments according to the disclosure will be described in detail with reference to the drawings. In the following embodiments, it is needless to mention that components (also including element steps and the like) thereof are not necessarily essential unless otherwise specified or unless clearly considered to be essential in principle.

### Test System 100

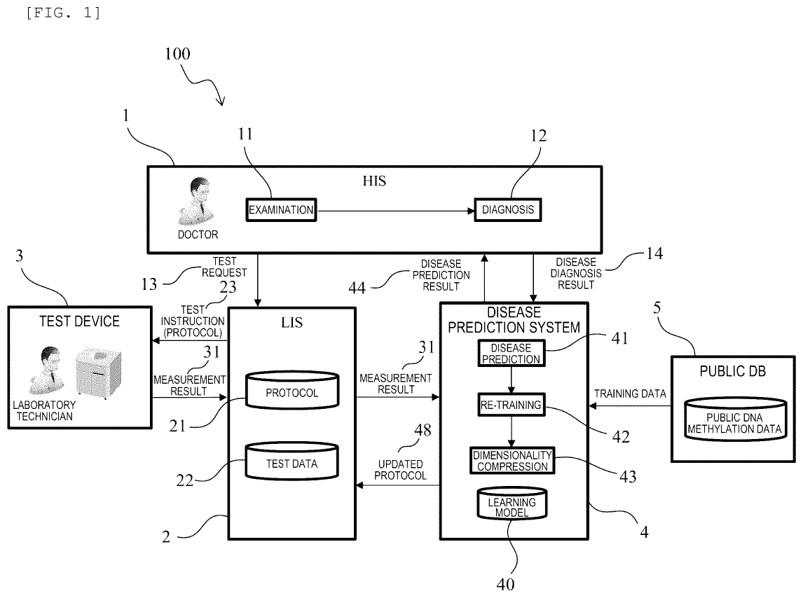

FIG. 1 is a diagram showing an overall configuration of a test system. In a test system 100, a doctor makes a test request of a specimen such as blood collected from a patient or the like, and performs diagnosis with reference to a test result of the specimen. The test system 100 includes a hospital information system (HIS) 1, a laboratory information system (LIS) 2, a test device 3, a disease prediction system 4, and a public database (DB) 5.

The HIS 1 is, for example, an electronic medical record, and a doctor makes a test request 13 of a specimen for the HIS 1. The doctor performs an examination 11 of a patient and a diagnosis 12 of a disease based on a disease prediction result received from the disease prediction system 4 described below.

The LIS 2 stores a protocol DB 21 and a test data DB 22. The protocol DB 21 stores data (protocol) defining a procedure for executing a test specified by a doctor. A laboratory technician performs a test of the specimen according to the protocol. The protocol is determined for each test. For example, a protocol for a DNA methylation test includes data indicating a location (a coordinate) where a DNA methylation level is to be measured. The test data DB 22 is a database that accumulates a measurement result 31 obtained by the test device 3. Upon receiving the test request 13 from the HIS 1, the LIS 2 issues a test instruction 23 and the protocol to the laboratory technician.

Upon receiving the test instruction 23 from the LIS 2, the laboratory technician performs a test on the specimen using the test device 3 according to the protocol. The test device 3 is, for example, a DNA sequencer and is a device for automatically decoding a nucleotide sequence of a DNA carrying genetic information of living organisms. The test device 3 can measure methylation levels of a plurality of locations (a plurality of coordinates) specified by a protocol. The measurement result 31 obtained by the test device 3 is stored in the test data DB 22. The LIS 2 transmits the measurement result 31 received from the test device 3 to the disease prediction system 4.

The disease prediction system 4 includes a learning model 40 obtained by performing machine learning using public DNA methylation data stored in the public DB 5. The learning model 40 receives the measurement result 31 obtained by the test device 3 and outputs a disease prediction result 44. The disease prediction result 44 output by the learning model 40 is transmitted to the HIS 1. In the disease prediction system 4, disease prediction 41 by the learning model 40, re-training 42 of the learning model 40, and dimensionality compression 43 that narrows down coordinates where a DNA methylation level is measured are repeatedly executed.

The doctor performs the diagnosis 12 with reference to the disease prediction result 44 transmitted from the disease prediction system 4. For example, the doctor performs an additional examination 11 with reference to the disease prediction result 44 transmitted from the disease prediction system 4 and determines the disease. The disease registered in the HIS 1 is transmitted to the disease prediction system 4 as a disease diagnosis result 14. A training data set in which the disease diagnosis result 14 and the measurement result 31 obtained by the test device 3 are associated with each other is used for the re-training 42 and the dimensionality compression 43 in the disease prediction system 4.

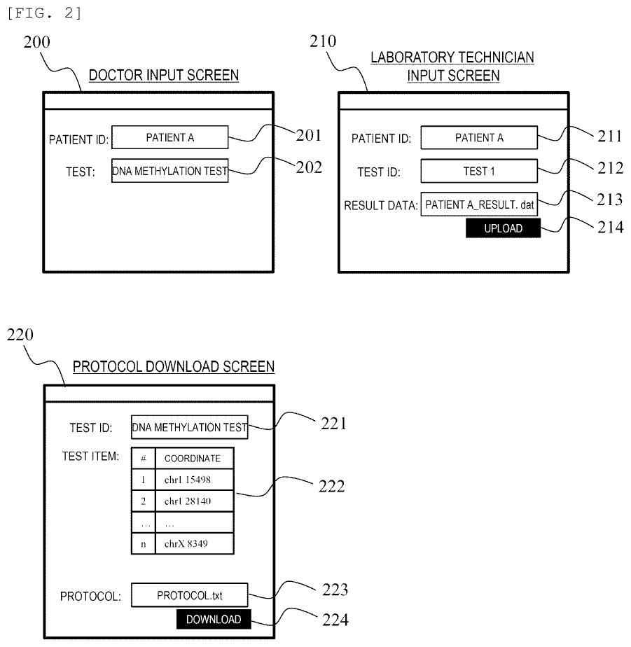

FIG. 2 is a diagram showing a GUI of the test system.

### Doctor Input Screen 200

The doctor makes the test request 13 via a doctor input screen 200 displayed on a display unit of the HIS 1. The doctor input screen 200 includes a patient ID input field 201 for inputting a patient ID for identifying a patient and a test input field 202 for indicating a content of a test to be requested.

### Laboratory Technician Input Screen 210

The laboratory technician uploads the measurement result 31 output by the test device 3 to the LIS 2 via a laboratory technician input screen 210. The laboratory technician input screen 210 is a screen displayed on a display unit of a computer communicably connected to the LIS 2 and the test device 3 or a display unit of the test device 3. The laboratory technician input screen 210 includes a patient ID input field 211 for inputting the patient ID for identifying the patient, a test ID input field 212 for inputting a test ID for identifying a test performed by the test device 3, a file specification field 213 for specifying a file including the measurement result 31 output by the test device 3, and an upload button 214 for uploading the specified file or the like to the LIS 2.

### Protocol Download Screen 220

A protocol download screen 220 is a screen for downloading a protocol indicating a location (a coordinate) where the DNA methylation level is to be measured to the LIS 2 or the test device 3. The protocol download screen 220 is a screen displayed on a display unit of the LIS 2, a display unit of the test device 3, or a display unit of a computer communicably connected to the test device 3. The protocol download screen 220 includes a test input field 221 indicating the content of a test, a test item display field 222 indicating a test item input in the test input field 221, a protocol field 223 indicating a file name of the protocol including the test item displayed in the test item display field 222, and a download button 224 for downloading a file with a file name specified in the protocol field 223 to the LIS 2 or the test device 3. The test items displayed in the test item display field indicate, for example, narrowed locations (coordinates) where the DNA methylation level is measured.

### Disease Prediction System 4

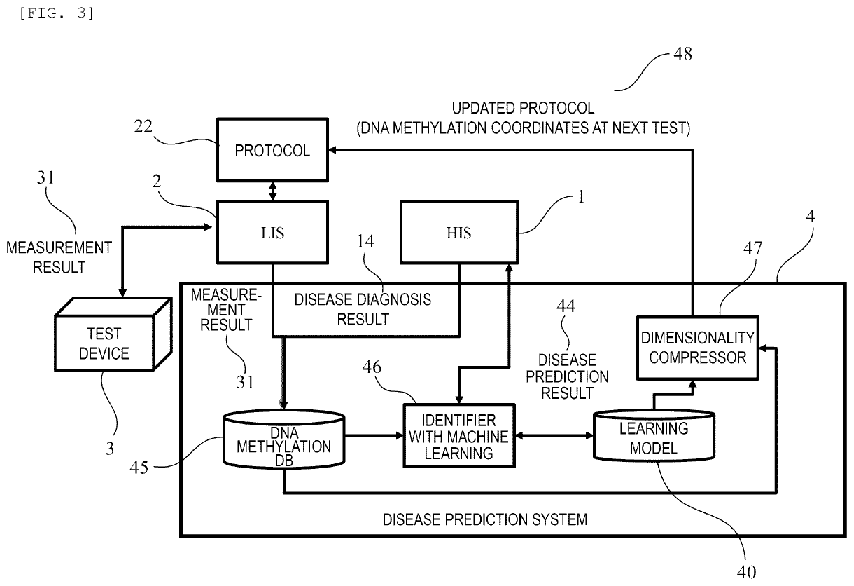

FIG. 3 is a software block diagram of the disease prediction system 4. The disease prediction system 4 includes the learning model 40, a DNA methylation DB 45, an identifier 46 based on machine learning, and a dimensionality compressor 47.

The DNA methylation DB 45 stores the public DNA methylation data stored in the public DB 5 and a training data set in which the disease diagnosis result 14 and the measurement result 31 obtained by the test device 3 are associated with each other. Every time the measurement by the test device 3 is performed and the disease diagnosis result 14 obtained by a doctor is associated with the measurement result 31 obtained by the test device 3, the number of training data sets stored in the DNA methylation DB 45 increases.

The identifier 46 based on machine learning is a program for predicting a disease using the trained or re-trained learning model 40. The identifier 46 outputs the disease prediction result 44 to the HIS 1.

In an initial stage, the learning model 40 performs machine learning using the public DNA methylation data stored in the public DB 5. Then, in the re-training stage, machine learning is performed using the training data set stored in the DNA methylation DB 45.

The dimensionality compressor 47 performs the dimensionality compression based on an analysis result of principal component analysis of the measurement result 31, cumulative contribution ratios of principal components, and a factor loading at each coordinate. The dimensionality compression can narrow down the locations (coordinates) where the DNA methylation level is to be measured. The dimensionality compressor 47 uploads an updated protocol 48 (the narrowed-down locations (coordinates) where the DNA methylation level is to be measured) to the protocol DB 21 of the LIS 2. The narrowed-down locations (coordinates) where the DNA methylation level is to be measured serve as locations (coordinates) measured at the next DNA methylation test.

### Hardware of Disease Prediction System 4

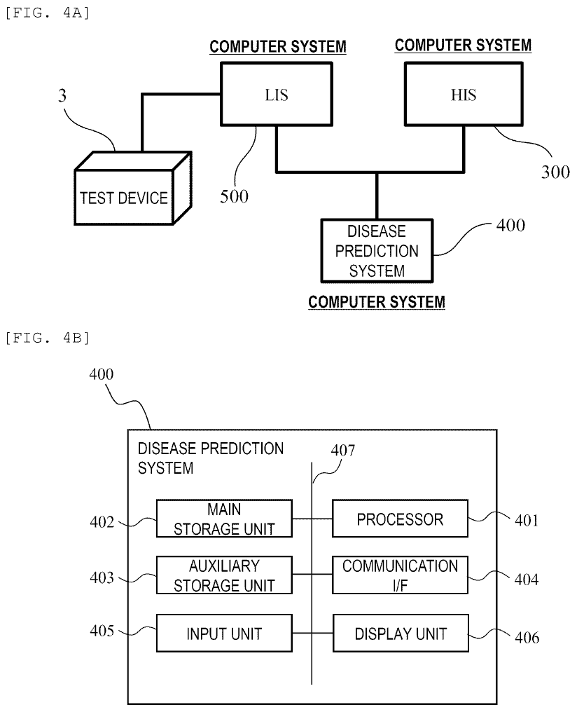

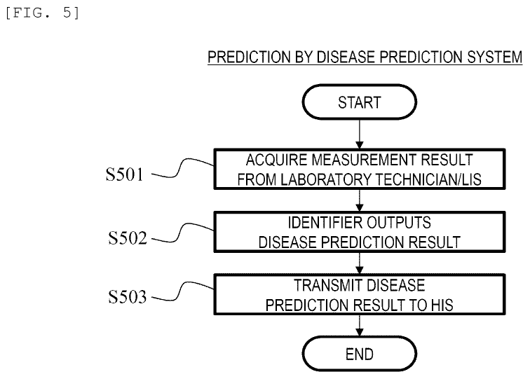

FIG. 4A is a diagram showing a hardware structure of the test system. The HIS 1, the LIS 2, and the disease prediction system 4 respectively include computer systems 300, 500, and 400 such as a server and a personal computer. The computer system 300 of the HIS 1, the computer system 500 of the LIS 2, and the computer system 400 of the disease prediction system 4 are communicably connected to each other via a network. The LIS 2 may be communicably connected to the test device 3, or may be communicably connected to a computer capable of communicating with the test device 3.

FIG. 4B is a hardware block diagram of the computer system of the disease prediction system. The computer system 400 includes a processor 401, a main storage unit 402, an auxiliary storage unit 403, a communication interface (reception unit, transmission unit) 404, an input unit 405, a display unit 406, and a bus 407 that communicably connects the above-described units.

The processor 401 is a central processing device that performs control of an operation of each unit of the disease prediction system 4. The processor 401 is, for example, a central processing unit (CPU), a digital signal processor (DSP), or an application specific integrated circuit (ASIC). The processor 401 loads a program stored in the auxiliary storage unit 403 to a work area of the main storage unit 402 in an executable manner. The main storage unit 402 stores a program executed by the processor 401, data processed by the processor, and the like. The main storage unit 402 is a flash memory, a random access memory (RAM), or the like. The auxiliary storage unit 403 stores various programs and various kinds of data. The auxiliary storage unit 403 stores, for example, an operating system (OS), various programs (for example, the learning model 40, the identifier 46, and the dimensionality compressor 47), and various kinds of data (for example, the DNA methylation DB 45). The auxiliary storage unit 403 is a solid state drive (SSD) device, a hard disk drive (HDD) device, or the like.

The communication I/F 404 communicates with the HIS 1 and the LIS 2, which are external devices, via a network. Specifically, the communication I/F 404 receives, from the LIS 2, the measurement result 31 obtained by the test device 3, transmits, to the HIS 1, the disease prediction result 44 output by the identifier 46, or transmits the updated protocol 48 (locations (coordinates) where the DNA methylation level is to be measured and which are narrowed down by the dimensionality compressor 47) to the LIS 2. The input unit 405 is a keyboard, a mouse, or the like, and the display unit 406 is a liquid crystal display device or the like.

### Prediction by Disease Prediction System 4

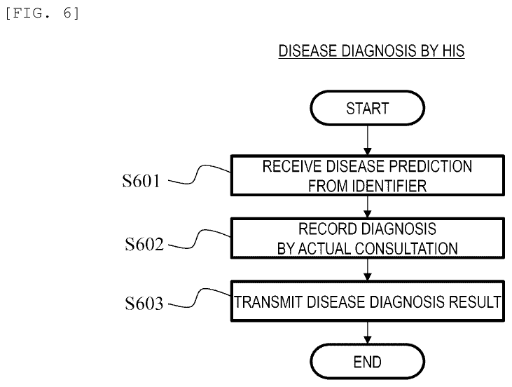

FIG. 5 is a flowchart of disease prediction by the disease prediction system. Each step in the flowchart of FIG. 5 is executed by the computer system of the disease prediction system 4. The disease prediction system 4 acquires the measurement result 31 obtained by the test device 3 from the laboratory technician (the test device 3 or the computer of the laboratory technician connected to the test device 3) or the LIS 2 (step S501). The identifier 46 outputs the disease prediction result 44 based on the input measurement result 31 (step S502). Then, the disease prediction system 4 transmits, to the HIS 1, the disease prediction result 44 output by the identifier 46 (step S503).

### Disease Diagnosis by HIS 1

FIG. 6 is a flowchart of disease diagnosis by the HIS. Each step in the flowchart of FIG. 6 is executed by the computer system of the HIS 1. The HIS 1 receives the disease prediction result 44 output by the identifier 46 (step S601). The doctor performs diagnosis by performing additional examination or the like with reference to the disease prediction result 44 received by the HIS 1, and records the diagnosis of the patient in the HIS 1 (step S602). Then, the HIS 1 transmits the recorded diagnosis (the disease diagnosis result 14) to the disease prediction system 4 (step S603).

### Dimensionality Compression by Disease Prediction System 4

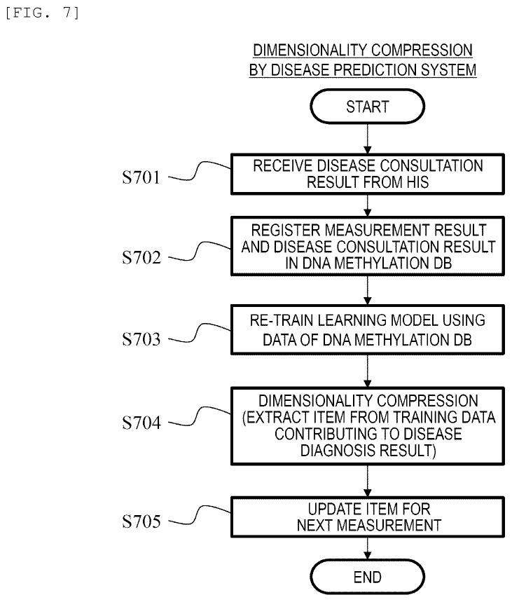

FIG. 7 is a flowchart of dimensionality compression by the disease prediction system. Each step in the flowchart of FIG. 7 is executed by the computer system of the disease prediction system 4. The disease prediction system 4 receives the disease diagnosis result 14 from the HIS 1 (step S701). Then, the disease prediction system 4 registers the measurement result 31 obtained by the test device 3 and the disease diagnosis result 14 related to the measurement result 31, which are in association with each other, in the DNA methylation DB 45 (step S702). The disease prediction system 4 re-trains the learning model 40 using data of the DNA methylation DB 45 (step S703). Accordingly, an updated learning model is generated. The processing up to here is the re-training of the learning model 40.

Next, the disease prediction system 4 performs dimensionality compression (step S704). The disease prediction system 4 extracts an item contributing to the disease diagnosis result 14 from the training data (the data of the DNA methylation DB 45) used for the re-training. The item is, for example, a location (a coordinate) where the DNA methylation level contributing to the disease diagnosis result 14 is to be measured. Then, the disease prediction system 4 uploads the updated protocol 48, indicating an item to be measured by the test device 3 from the next time on, to the LIS 2 to update a protocol in the protocol DB 21 (step S705).

### Learning Example

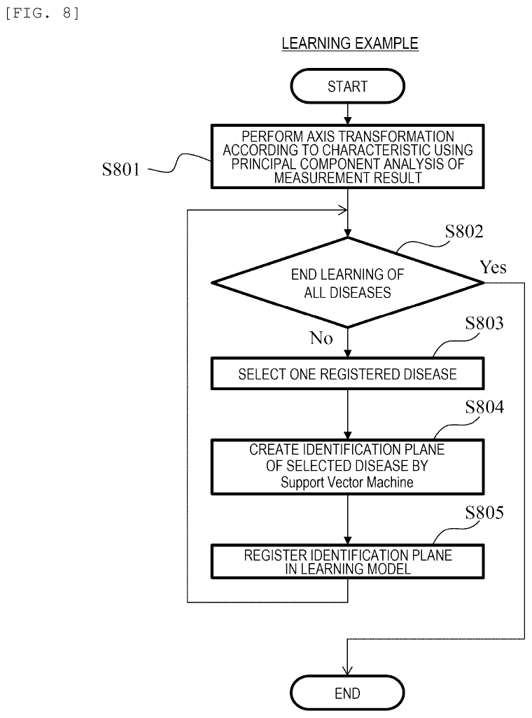

FIG. 8 is a flowchart showing an example of learning by the disease prediction system. First, the disease prediction system 4 performs principal component analysis of the measurement result 31 stored in the DNA methylation DB 45 and performs axis transformation according to a characteristic of the measurement result 31 (step S801). With the principal component analysis, the training data used in the machine learning performed by the learning model 40 can be made easy to handle.

The disease prediction system 4 determines whether learning of all diseases has ended (step S802). All diseases are all the diseases identified by the identifier 46, and for example, when the identifier 46 identifies stomach cancer, lung cancer, colon cancer, and the like from the measurement result 31 obtained by the test device 3, all diseases refer to gastric cancer, lung cancer, colon cancer, and the like. In an initial stage of the learning, of course, learning of all diseases is not completed (step S802: No), and therefore, the disease prediction system 4 selects one disease from all registered diseases (step S803).

Then, the disease prediction system 4 creates an identification plane of the disease selected in step S803 using a support vector machine (step S804). The disease prediction system 4 creates the identification plane for identifying the selected disease based on the measurement result 31 subjected to the supervised (disease diagnosis result 14) axis transformation. Then, the disease prediction system 4 registers the created identification plane in the learning model 40 (step S805).

The disease prediction system 4 repeats steps S803 to S805 until the identification planes for all diseases are registered in the learning model 40. When the identification planes for all diseases are registered in the learning model 40 (step S802: Yes), the learning is ended.

### Dimensionality Compression Example

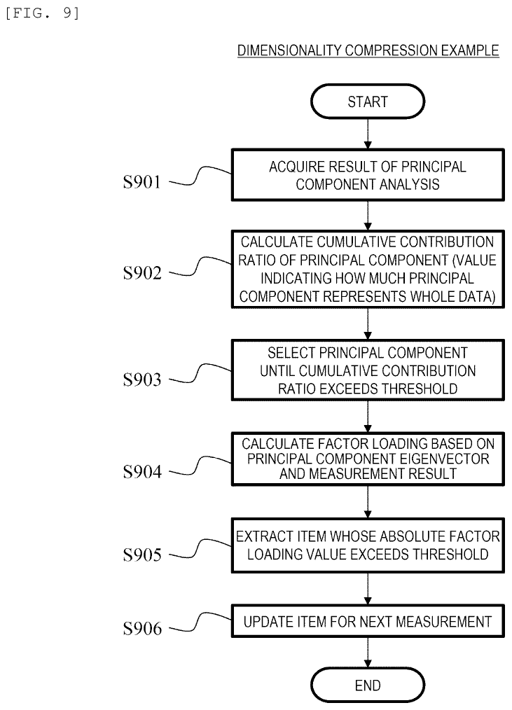

FIG. 9 is a flowchart showing an example of dimensionality compression by the disease prediction system. The disease prediction system 4 acquires a result of the above-described principal component analysis (step S901). The disease prediction system 4 calculates a contribution ratio of each principal component by a known method and calculates a cumulative contribution ratio (step S902). The cumulative contribution ratio is a value indicating how much the principal component represents the entire data. For example, when a contribution ratio of a first principal component 1 is α, a contribution ratio of a second principal component 2 is β, and a contribution ratio of a third principal component 3 is γ, the cumulative contribution ratio is α+β+γ. Then, the disease prediction system 4 selects a principal component until the cumulative contribution ratio obtained by sequentially adding the contribution ratio of each principal component from the first principal component 1 exceeds a threshold (for example, 0.9) (step S903).

Next, the disease prediction system 4 calculates a factor loading (degree of correlation) based on an eigenvector of a principal component and the measurement result 31 by a known method (step S904). Here, a factor loading is calculated for each of the plurality of selected principal components. The factor loading indicates the degree of correlation of coordinates, where the DNA methylation level is measured, with the principal component. Then, the disease prediction system 4 extracts an item whose absolute factor loading exceeds the threshold (step S905). Then, the disease prediction system 4 uploads the updated protocol 48 indicating the extracted item (item (coordinate) to be measured by the test device 3 from the next time on) to the LIS 2 to update the protocol in the protocol DB 21 (step S906).

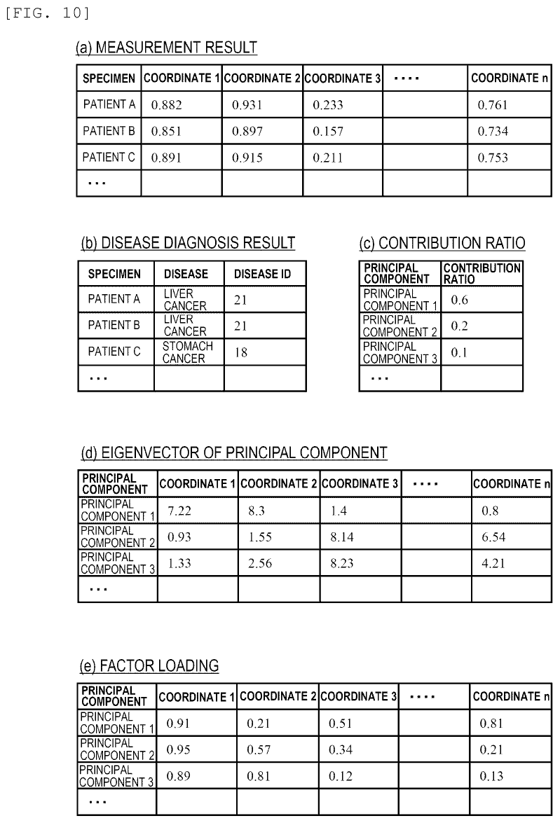

FIG. 10 is a diagram showing an example of each data. Details of each data will be described with reference to FIG. 10. The specific example of each data shown in FIG. 10 is an example.

### Measurement Result 31

The measurement result 31 shown in (a) of FIG. 10 is data indicating DNA methylation levels at coordinates 1 to n of each patient measured by the test device 3. An object of the disclosure is to reduce the number of locations (coordinates) where the DNA methylation level is to be measured, when performing a DNA methylation test. The measurement result 31 is a value between 0 and 1, and a larger value means a higher DNA methylation level.

### Disease Diagnosis Result 14

The disease diagnosis result 14 shown in (b) of FIG. 10 is data in which a disease diagnosed by a doctor is registered for each patient. The doctor registers the diagnosed disease in the HIS 1 for each patient. The disease diagnosis result 14 registered in the HIS 1 is transmitted to the disease prediction system 4 and is used to re-train the learning model 40. In the disease diagnosis result 14 shown in (c) of FIG. 10, a disease name and a disease ID indicating the disease are registered for each patient.

### Contribution Ratio

The contribution ratio shown in (c) of FIG. 10 is data indicating a contribution ratio of each principal component obtained by the principal component analysis. The contribution ratio is calculated by a known method. In the example shown in (c) of FIG. 10, when the threshold of the cumulative contribution ratio in step S903 is 0.9, a principal component A (contribution ratio=0.6), a principal component B (contribution ratio=0.2), and a principal component C (contribution ratio=0.1) are selected until the total of the contribution ratios (cumulative contribution ratio) exceeds the threshold.

### Eigenvector Data of Principal Component

The eigenvector data of the principal component shown in (b) of FIG. 10 is data of eigenvectors at the coordinates 1 to n of each principal component obtained by the principal component analysis.

### Factor Loading

The factor loading shown in (e) of FIG. 10 is data indicating a factor loading of each principal component calculated by the principal component analysis. The factor loading is a value calculated based on the above-described eigenvector and indicates a correlation with the principal component. In the disclosure, an item (coordinate) whose absolute factor loading (for example, 0.8) exceeds a threshold is output as an item (coordinate) to be measured from the next time on.

### Selection of Principal Component

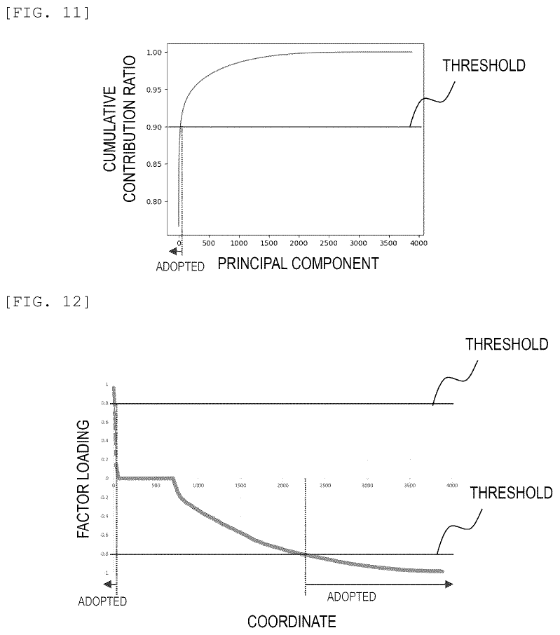

FIG. 11 is a graph showing a cumulative contribution ratio. The horizontal axis represents a principal component, and the vertical axis represents a cumulative contribution ratio. In the example shown in FIG. 11, the threshold is 0.9, and the principal component is selected until the cumulative contribution ratio exceeds 0.9. In the example shown in FIG. 11, the principal component in the range indicated as being adopted is selected.

### Selection of Coordinate

FIG. 12 is a graph showing a factor loading. The horizontal axis represents the coordinate, and the vertical axis represents the factor loading. In the example shown in FIG. 12, the absolute value of the threshold is 0.8, and coordinates where the absolute factor loading exceeds 0.8 are selected. In the example shown in FIG. 12, the coordinates in the range indicated as being adopted are selected. In the graph shown in FIG. 12, the coordinates are sorted in the order of magnitude of the factor loading, and then plotted along the horizontal axis from a coordinate having the largest factor loading.

### Effects of Embodiments

By selecting a principal component based on a cumulative contribution ratio and extracting a coordinate correlated with a principal component selected based on a factor loading, locations (coordinates) where the DNA methylation level is to be measured can be narrowed down in the DNA methylation level measurement method. As a result, in the test for measuring the DNA methylation level, the lengthening of measurement and the increase in measurement cost can be prevented.

By selecting the principal component based on the cumulative contribution ratio, the locations (coordinates) where the DNA methylation level is to be measured can be narrowed down without reducing the information content of the measurement result.

Coordinates correlated with the principal component can be extracted based on the factor loading, and the methylation level can be measured at the disease-related coordinates.

The coordinate as a methylation level measurement target can be set as a protocol in the test device 3, and therefore, the methylation level at a desired coordinate can be easily measured.

By executing the principal component analysis in the re-training of the learning model 40, over-training can be prevented, and the principal component analysis result can be used in dimensionality compression.

The disclosure is not limited to the above-described embodiments, and includes various modifications. The above-described embodiments have been described in detail to facilitate understanding of the disclosure, and the disclosure is not necessarily limited to those including all the configurations described above. A part of a configuration according to the embodiment may be added to, deleted from, or replaced with another configuration.

For example, although an example of identifying a cancer type has been described in the above embodiment, the disclosure is not limited to identification of a cancer type, and is applicable to identification of various diseases such as Alzheimer's disease and lifestyle disease.

### REFERENCE SIGNS LIST

100: test system
1: HIS
13: test request
14: disease diagnosis result
2: LIS
21: protocol DB
22: test data DB
23: test instruction
3: test device
31: measurement result
4: disease prediction system
40: learning model
44: disease prediction result
45: DNA methylation DB
46: identifier
47: dimensionality compressor
48: updated protocol
5: public DB
300, 400, 500: computer system
401: processor
402: main storage unit
403: auxiliary storage unit
404: communication interface
405: input unit
406: display unit

1. A DNA methylation level measurement method, comprising:
performing machine learning using a training data set including a measurement result of a DNA methylation level at a plurality of coordinates and information on a disease corresponding to the measurement result to generate a learning model configured to predict a disease;
selecting one or a plurality of principal components of the measurement result;
calculating a degree of correlation indicating a correlation between the selected one or plurality of principal components and the plurality of coordinates; and
determining, based on the calculated degree of correlation, a coordinate to be measured in a test for measuring the DNA methylation level, from among the plurality of coordinates.

2. The DNA methylation level measurement method according to claim 1, wherein
the selection of the principal component includes calculating a contribution ratio of each of the plurality of principal components and selecting the one or plurality of principal components based on a cumulative contribution ratio which is a sum of contribution ratios obtained by sequentially adding the contribution ratio of each principal component from a first principal component.

3. The DNA methylation level measurement method according to claim 1, wherein
the degree of correlation is a factor loading indicating the correlation of the plurality of coordinates with respect to the one or plurality of principal components.

4. The DNA methylation level measurement method according to claim 1, further comprising:
transmitting a protocol including the determined coordinate to be measured to a test device configured to measure the DNA methylation level at a coordinate designated by the protocol.

5. The DNA methylation level measurement method according to claim 1, wherein
the generation of the learning model includes executing principal component analysis on the measurement result.

6. A disease prediction system comprising:
a reception unit configured to receive a measurement result of a DNA methylation level at a plurality of coordinates and information on a disease corresponding to the measurement result; and
a computer system, wherein
the computer system
performs machine learning using a training data set including the measurement result received by the reception unit and the information on the disease corresponding to the measurement result to generate a learning model configured to predict a disease,
selects one or a plurality of principal components of the measurement result,
calculates a degree of correlation indicating a correlation between the selected one or plurality of principal components and the plurality of coordinates, and
determines, based on the calculated degree of correlation, a coordinate to be measured in a test for measuring the DNA methylation level, from among the plurality of coordinates.

7. The disease prediction system according to claim 6, wherein
the computer system calculates a contribution ratio of each of the plurality of principal components and selects the one or plurality of principal components based on a cumulative contribution ratio which is a sum of contribution ratios obtained by sequentially adding the contribution ratio of each principal component from a first principal component.

8. The disease prediction system according to claim 6, wherein
the degree of correlation is a factor loading indicating the correlation of the plurality of coordinates with respect to the one or plurality of principal components.

9. The disease prediction system according to claim 6, further comprising:
a transmission unit configured to transmit a protocol including the determined coordinate to be measured to a test device configured to measure the DNA methylation level at a coordinate designated by the protocol.

10. The disease prediction system according to claim 6, wherein
the computer system executes principal component analysis on the measurement result and performs machine learning using a training data set including the measurement result which is axis-transformed by the principal component analysis and the information on the disease corresponding to the measurement result.

11. A test system comprising:
a test device configured to perform a measurement of a DNA methylation level at a plurality of coordinates ;
a disease prediction system including a learning model configured to predict a disease based on the measurement result of the DNA methylation level at the plurality of coordinates, the measurement result being obtained by the test device; and
a hospital information system configured to receive a disease prediction result from the disease prediction system and output a disease diagnosis result, wherein
the disease prediction system
receives the disease diagnosis result from the hospital information system,
re-trains the learning model using a training data set including the measurement result and the disease diagnosis result,
selects one or a plurality of principal components of the measurement result,
calculates a degree of correlation indicating a correlation between the selected one or plurality of principal components and the plurality of coordinates, and
determines, based on the calculated degree of correlation, a coordinate to be measured in a test for measuring the DNA methylation level, from among the plurality of coordinates.

12. The test system according to claim 11, wherein
the disease prediction system calculates a contribution ratio of each of the plurality of principal components and selects the one or plurality of principal components based on a cumulative contribution ratio which is a sum of contribution ratios obtained by sequentially adding the contribution ratio of each principal component from a first principal component.

13. The test system according to claim 11, wherein
the degree of correlation is a factor loading indicating the correlation of the plurality of coordinates with respect to the one or plurality of principal components.

14. The test system according to claim 11, further comprising:
a transmission unit configured to transmit a protocol including the determined coordinate to be measured to a test device configured to measure the DNA methylation level at a coordinate designated by the protocol.

15. The test system according to claim 11, wherein
the disease prediction system executes principal component analysis on the measurement result and performs machine learning using a training data set including the measurement result which is axis-transformed by the principal component analysis and the information on the disease corresponding to the measurement result.
